(19) United States
(12) Patent Application Publication
(10) Pub. No.: US 2007/0043006 A1
(43) Pub. Date: Feb. 22, 2007

(54) Formulations of non-steroidal anti-inflammatory agents to treat pathologic ocular angiogenesis

(76) Inventor: David P. Bingaman, Weatherford, TX (US)

Correspondence Address: Teresa J Schultz;Alcon Research, 6201 South Freeway, Q-148, Forth Worth, TX, 76134-2099, US

(21) Appl. No.: 10/557,133

(22) PCT Filed: Jun. 14, 2004
(86) PCT No.: PCT/US04/18792

§ 371 Date: Nov. 17, 2005

(30) Foreign Application Priority Data
Jun. 13, 2003 (US) 60/478252
Jun. 13, 2003 (US) 60/478227

(51) Int. Cl. A61K 31/573; A61K 31/192

(52) U.S. Cl. 514/171; 514/569

(57) ABSTRACT

Methods for the use of NSAIs in combination with anecortave acetate are disclosed for preventing and treating pathologic ocular angiogenesis and associated edema, retinal edema, PPDR or NPDR.

This application claims priority from U.S. Ser. No. 60/478,227, filed Jun. 13, 2003 and U.S. Ser. No. 60/478,252, filed Jun. 13, 2003.

The present invention is directed to the prevention and treatment of eye diseases characterized by pathologic ocular angiogenesis and/or retina or subretinal edema. In particular, the present invention is directed to the use of certain formulations of non-steroidal anti-inflammatories (NSAIs) alone and in combination with anecortave acetate to treat such ocular angiogenesis and associated retina or subretinal edema

### BACKGROUND OF THE INVENTION

There are many agents known to inhibit the formation of new blood vessels (angiogenesis or neovascularization). For example, steroids functioning to inhibit angiogenesis in the presence of heparin or specific heparin fragments are disclosed in Crum, et al., A New Class of Steroids Inhibits Angiogenesis in the Presence of Heparin or a Heparin Fragment, Science, Vol. 230:1375-1378, Dec. 20, 1985. The authors refer to such steroids as “angiostatic” steroids. Included within this class of steroids found to be angiostatic are the dihydro and tetrahydro metabolites of cortisol and cortexolone. In a follow-up study directed to testing a hypothesis as to the mechanism by which the steroids inhibit angiogenesis, it was shown that heparin/angiostatic steroid compositions cause dissolution of the basement membrane scaffolding to which anchorage dependent endothelia are attached resulting in capillary involution; see, Ingber, et al., A Possible Mechanism for Inhibition of Angiogenesis by Angiostatic Steroids: Induction of Capillary Basement Membrane Dissolution, Endocrinology, Vol. 119:1768-1775, 1986.

A group of tetrahydro steroids useful in inhibiting angiogenesis is disclosed in U.S. Pat. No. 4,975,537, Aristoff, et al. The compounds are disclosed for use in treating head trauma, spinal trauma, septic or traumatic shock, stroke, and hemorrhage shock. In addition, the patent discusses the utility of these compounds in embryo implantation and in the treatment of cancer, arthritis, and arteriosclerosis. Some of the steroids disclosed in Aristoff et al. are disclosed in U.S. Pat. No. 4,771,042 in combination with heparin or a heparin fragment for inhibiting angiogenesis in a warm blooded animal.

Compositions of hydrocortisone, “tetrahydrocortisol-S,” and U-72,745G, each in combination with a beta cyclodextrin, have been shown to inhibit corneal neovascularization: Li, et al., Angiostatic Steroids Potentiated by Sulphated Cyclodextrin Inhibit Corneal Neovascularization, Investigative Ophthalmology and Visual Science, Vol. 32(11):2898-2905, October, 1991. The steroids alone reduce neovascularization somewhat, but are not effective alone in effecting regression of neovascularization.

Tetrahydrocortisol (IF) has been disclosed as an angiostatic steroid in Folkman, et al., Angiostatic Steroids, Am. Surg., Vol. 206(3), 1987, wherein it is suggested angiostatic steroids may have potential use for diseases dominated by abnormal neovascularization, including diabetic retinopathy, neovascular glaucoma, and retrolental fibroplasia.

Exudative or wet age-related macular degeneration (AMD) and proliferative diabetic retinopathy (PDR) are characterized by pathologic ocular angiogenesis and are the most common causes of acquired blindness in developed countries. Abnormal new blood vessel growth in exudative AMD is derived from the choriocapillaris underneath the retina pigmented epithelium (RPE) and neurosensory retina. The new vessel formation is termed choroidal neovascularization or CNV. This type of angiogenesis can grow through Bruch's membrane and enter the potential space between the RPE and photoreceptors. Often the fragile CNV leaks fluid, blood components and can lead to frank hemorrhage. Thus, during CNV, fluid accumulation is termed subretinal. In contrast, abnormal new blood vessel growth in PDR emminates from the retinal capillaries and grows from the inner retina into the vitreous humor, i.e., preretinal NV. As in exudative AMD, these pathologic vessels can leak fluid and lead to intraretinal and vitreal hemorrhage. Moreover, diabetic patients may experience enhanced vascular permeability from the normal retinal capillaries leading a condition called macular edema

Diabetes mellitus is characterized by persistent hyperglycemia that produces reversible and irreversible pathologic changes within the microvasculature of various organs. Diabetic retinopathy (DR), therefore, is a retinal microvascular disease that is manifested as a cascade of stages with increasing levels of severity and worsening prognoses for vision. Some major risk factors reported for developing diabetic retinopathy include the duration of diabetes mellitus, quality of glycemic control, and presence of systemic hypertension. DR is broadly classified into 2 major clinical stages: nonproliferative diabetic retinopathy (NPDR) and proliferative diabetic retinopathy (PDR), where the term “proliferative” refers to the presence of preretinal neovascularization (NV). NPDR encompasses a range of clinical subcategories which include initial “background” DR, where small multifocal changes are observed within the retina (e.g., microaneurysins, “dot-blot” hemorrhages, and nerve fiber layer infarcts), through preproliferative DR, which immediately precedes the development of preretinal NV. Diabetic macular edema can be seen during either NPDR or PDR, however, it often is observed in the latter stages of NPDR and is a prognostic indicator of progression towards development of the most severe stage, PDR.

Macular edema is the major cause of vision loss in diabetic patients, whereas preretinal neovascularization (PDR) is the major cause of legal blindness. NPDR and subsequent macular edema are associated, in part, with retinal ischemia that results from the retinal microvasculopathy induced by persistent hyperglycemia. Data accumulated from animal models and empirical human studies show that retinal ischemia is often associated with increased local levels of proinflammatory and/or proangiogenic growth factors and cytokines, such as prostaglandin E₂, vascular endothelial growth factor (VEGF), insulin-like growth factor-1 (IGF-1), etc. These molecules can alter the retinal microvasculature and cause pathologic changes such as capillary extracellular matrix remodeling, retinal vascular leakage leading to edema, and angiogenesis.

Today, no pharmacologic therapy is approved for the treatment of DR and/or macular edema. The current standard of care is laser photocoagulation, which is used to stabilize or resolve macular edema and retard the progression toward preretinal NV. Laser photocoagulation may reduce retinal ischemia by destroying healthy tissue and thereby decrease metabolic demand; it also may modulate the expression and production of various cytokines and trophic factors. Unfortunately, laser photocoagulation is a cytodestructive procedure and the visual field of the treated eye is irreversibly compromised. Other than diabetic macular edema, retinal edema can be observed in various other posterior segment diseases, such as posterior uveitis, branch retinal vein occlusion, surgically induced inflammation, endophthalmitis (sterile and non-sterile), scleritis, and episcleritis, etc.

Glucocorticoids have been used by the medical community to treat certain disorders of the back of the eye, in particular: Kenalog (triamcinolone acetonide), Celestone Soluspan (betamethasone sodium phosphate), Depo-Medrol (methylprednisolone acetate), Decadron (dexamethasone sodium phosphate), Decadron L. A. (dexamethasone acetate), and Aristocort (triamcinolone diacetate). These products are commonly administered via a periocular injection for the treatment of inflammatory disorders. Because of the lack of efficacious and safe therapies, there is a growing interest in using glucocorticoids for the treatment of, for example, retinal edema and age-related macular degeneration (AMD) via intravitreal administration. Bausch & Lomb and Control Delivery Systems are evaluating fluocinolone acetonide delivered via an intravitreal implant for the treatment of macular edema. Oculex Pharmaceuticals is studying as intravitreal dexamethasone implant for persistent macular edema In addition, ophthalmologists are experimenting with intravitreal injection of Kenalog for the treatment of recalcitrant cystic diabetic macular edema and for exudative AMD.

Although glucocorticoids are very effective in treating many ocular conditions, there are significant side effects associated with the available products. Side effects include: endopthalmitis, cataracts, and elevated intraocular pressure (IOP). Although some side effects are due to the glucocorticoid itself, some may result from, or be exacerbated by, excipients in the formulations and the method of delivery.

The topical ocular use of NSAIs includes the maintenance of pupillary dilation during surgery, control of inflammation after cataract extraction and following argon laser trabeculoplasty. They are also used for non-surgically induced inflammatory disorders of the eye, such as, allergic conjunctivitis and pain following radial keratotomy or excimer laser procedures. Several topical ocular formulations are available: flurbiprofen (Ocufen®, Allergan), diclofenac (Voltaren®, Ciba Vision), and Ketorolac (Acular®, Allergan), see Ophthalmic Drug Facts, 1999, pp. 82-83 and 90-93.

There is a need for NSAI formulations that are effective in treating pathologic ocular neovascularization, specifically within the posterior segment, while causing no or lessened adverse reactions. Furthermore, there are no NSAIs developed for treating persons suffering from ocular edema and/or NPDP The formulations of this invention meet those needs.

### SUMMARY OF THE INVENTION

The present invention is directed to the prevention and treatment of diseases and disorders of the eye involving pathologic ocular angiogenesis using formulations of NSAIs alone and in combination with anecortave acetate. The present invention is further directed to the use of NSAIs for treating persons suffering from retinal edema and/or NPDR.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1. Local administration of anecortave acetate inhibits preretinal neovascularization (neovascular score) in a rat model of oxygen-induced retinopathy.

### DETAILED DESCRIPTION OF THE INVENTION

Posterior segment neovascularization (NV) is the vision-threatening pathology responsible for the two most common causes of acquired blindness in developed countries: exudative age-related macular degeneration (AMD) and proliferative diabetic retinopathy (PDR). Currently the only approved treatments for posterior segment NV that occurs during exudative AMD is laser photocoagulation or photodynamic therapy with Visudyne®; both therapies involve occlusion of affected vasculature which results in localized laser-induced damage to the retina. For patients with PDR, surgical interventions with vitrectomy and removal of preretinal membranes are the only options currently available. No strictly pharmacologic treatment has been approved for use against posterior segment NV, although several different compounds are being evaluated clinically, including, for example, anecortave acetate (Alcon Research, Ltd.), EYE 001 (Eyetech), and rhuFabV2 (Genentech) for AMD and LY333531 (Lilly) and Fluocinolone (Bausch & Lomb) for exudative AMD and/or diabetic macular edema.

Pathologic ocular angiogenesis, which includes posterior segment NV, occurs as a cascade of events that progress from an initiating stimulus to the formation of abnormal new capillaries. The inciting cause in both exudative AMD and PDR is still unknown, however, the elaboration of various proangiogenic growth factors appears to be a common stimulus. Soluble growth factors, such as vascular endothelial growth factor (VEGF), basic fibroblast growth factor (bFGF or FGF-2), insulin-like growth factor 1 (IGF-1), etc., have been found in tissues and fluids removed from patients with pathologic ocular angiogenesis. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. In AMD, fluid accumulation from hyperpermeable choroidal capillaries and CNV leads to edema within and/or under the retina, i.e., subretinal edema, where in DR, increased vascular permeability of the retinal capillaries leads to intraretinal edema Differentiation into a mature capillary is indicated by the presence of a continuous basement membrane and normal endothelial junctions between other endothelial cells and pericytes; however, this differentiation process is often impaired during pathologic conditions.

An effective pharmacologic therapy for pathologic ocular angiogenesis and any associated edema would provide substantial efficacy to the patient, thereby avoiding invasive surgical or damaging laser procedures. Effective treatment of the pathologic ocular angiogenesis and edema would improve the patient's quality of life and productivity within society. Also, societal costs associated with providing assistance and health care to the blind could be dramatically reduced.

According to the methods of the present invention, a composition comprising a NSAI alone or in combination with anecortave acetate in a pharmaceutically acceptable carrier for local administration is administered to a mammal in need thereof. The compositions are formulated in accordance with methods known in the art for the particular route of administration desired.

Preferred NSAIs for treating retinal edema, PPDR, and NPDR include all non-commercially and commercially available NSAIs suitable for ophthalmic use, including, but not limited to: amfenac, nepafenac, and related compounds as disclosed in commonly owned U.S. Pat. No. 5,475,034 and in U.S. Pat. No. 4,910,225 both of which are incorporated herein by reference, ketorolac, diclofenac, and flurbiprofen.

The formulations can be delivered by topical ocular administration, intravitreal, posterior juxtascleral, or subconjunctival injection as well as via an implanted device as further below described. All cited patents and publications are herein incorporated by reference.

Particularly preferred implanted devices include: various solid and semi-solid drug delivery implants, including both non-erodible, non-degradable implants, such as those made using ethylene vinyl acetate, and erodible or biodegradable implants, such as those made using polyanhydrides or polylactides. Drug delivery implants, particularly ophthalmic drug delivery implants are generally characterized by at least one polymeric ingredient. In many instances, drug delivery implants contain more than one polymeric ingredient.

For example, U.S. Pat. No. 5,773,019 discloses implantable controlled release devices for delivering drugs to the eye wherein the implantable device has an inner core containing an effective amount of a low solubility drug covered by a non-bioerodible polymer coating layer that is permeable to the low solubility drug.

U.S. Pat. No. 5,378,475 discloses sustained release drug delivery devices that have an inner core or reservoir comprising a drug, a first coating layer which is essentially impermeable to the passage of the drug, and a second coating layer which is permeable to the drug. The first coating layer covers at least a portion of the inner core but at least a small portion of the inner core is not coated with the first coating layer. The second coating layer essentially completely covers the first coating layer and the uncoated portion of the inner core.

U.S. Pat. No. 4,853,224 discloses biodegradable ocular implants comprising microencapsulated drugs for implantation into the anterior and/or posterior chambers of the eye. The polymeric encapsulating agent or lipid encapsulating agent is the primary element of the capsule.

U.S. Pat. No. 5,164,188 discloses the use of biodegradable implants in the suprachoroid of an eye. The implants are generally encapsulated. The capsule, for the most part, is a polymeric encapsulating agent. Material capable of being placed in a given area of the suprachoroid without migration, “such as oxycel, gelatin, silicone, etc.” can also be used.

U.S. Pat. No. 6,120,789 discloses the use of a non-polymeric composition for in situ formation of a solid matrix in an animal, and use of the composition as a medical device or as a sustained release delivery system for a biologically-active agent, among other uses. The composition is composed of a biocompatible, non-polymeric material and a pharmaceutically acceptable, organic solvent. The non-polymeric composition is biodegradable and/or bioerodible, and substantially insoluble in aqueous or body fluids. The organic solvent solubilizes the non-polymeric material, and has a solubility in water or other aqueous media ranging from miscible to dispersible. When placed into an implant site in an animal, the non-polymeric composition eventually transforms into a solid structure. The resulting implant provides a system for delivering a pharmaceutically effective active agent to the animal. According to the '789 patent, suitable organic solvents are those that are biocompatible, pharmaceutically acceptable, and will at least partially dissolve the non-polymeric material. The organic solvent has a solubility in water ranging from miscible to dispersible. The solvent is capable of diffusing, dispersing, or leaching from the composition in situ into aqueous tissue fluid of the implant site such as blood serum, lymph, cerebral spinal fluid (CSF), saliva, and the like. According to the '789 patent, the solvent preferably has a Hildebrand (HLB) solubility ratio of from about 9-13 (ca1/cm3)1/2 and it is preferred that the degree of polarity of the solvent is effective to provide at least about 5% solubility in water.

Polymeric ingredients in erodible or biodegradable implants must erode or degrade in order to be transported through ocular tissues and eliminated. Low molecular weight molecules, on the order of 4000 or less, can be transported through ocular tissues and eliminated without the need for biodegradation or erosion.

Another implantable device that can be used to deliver formulations of the present invention is the biodegradable implants described in U.S. Pat. No. 5,869,079.

For posterior juxtascleral delivery of a formulation of the present invention, the preferred device is disclosed in commonly owned U.S. Pat. No. 6,413,245 B1 (cannula). Other preferred devices for delivery are disclosed in other commonly owned patents and patent applications: U.S. Pat. Nos. 6,416,777 B1 and 6,413,540 B1 (device for implantation on outer surface of the sclera).

Exemplary NSAI formulations which serve the purpose of the present invention are specifically shown below in Examples 1-3. The formulations may be delivered as previously described. The formulations of the present invention can include a NSAI at a concentration of about 0.001 to 4, preferably 0.01 to 0.5, non-ionic surfactants, e.g., polysorbates, also known as Tweens, pluronics, and Spans. Ionic surfactants can also be used, e.g., sodium lauryl sulfate or anionic bile salts. Amphoteric surfactants, such as, lecithin and hydrogenated lecithin can be used. The pH can vary from 5.0-8.4, but is preferably about 6.8-7.8. Other appropriate buffer systems, such as, citrate or borate can be employed in the present formulations. Different osmolality adjusting agents can also be used, such as, potassium chloride, calcium chloride, glycerin, dextrose, or mannitol. Certain, but not all NSAIs can be dosed topically for the treatment of retinal edema, pre-proliferative diabetic retinopathy (PPDR) and/or nonproliferative diabetic retinopathy (NPDR), in particular, nepafenac.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

| | |
|---|---|
| Nepafenac | 0.01-0.5% |
| Polysorbate 80 | 0.01% |
| Benzalkonium Chloride | 0.01% + 10% excess |
| Disodium EDTA | 0.1% |
| Monobasic Sodium Phosphate | 0.03% |
| Dibasic Sodium Phosphate | 0.1% |
| Sodium Chloride | q.s. 290-300 mOsm/Kg |
| PH adjustment with NaOH and/or HCl | pH 4.2-7.4 |
| Water | q.s. 100% |

### EXAMPLE 2

| | |
|---|---|
| Nepafenac | 0.01-0.5% |
| Hydroxypropyl Methylcellulose | 0.5% |
| Polysorbate 80 | 0.01% |
| Benzalkonium Chloride | 0.01% + 5% excess |
| Disodium EDTA | 0.01% |
| Dibasic Sodium Phosphate | 0.2% |
| Sodium Chloride | q.s. 290-300 mOsm/Kg |
| PH adjustment with NaOH and/or HCl | pH 4.2-7.4 |
| Water | q.s. 100% |

The present invention also contemplates the use of NSAIs in combination with the angiostatic agent, anecortave acetate, or other angiostatic agents. As used herein, anecortave acetate refers to 4,9(11)-pregnadien-17α,21-diol-3,20dione-21-acetate and its corresponding alcohol (4,9(11)-pregnadiene-17α,21-diol-3,20-dione). Presently, anecortave acetate is undergoing clinical trials for its use in persons suffering from subfoveal choroidal neovascularization secondary to AMD. The anecortave acetate can be delivered, e.g., via juxtascleral injection in depots comprising 3-30 mg of anecortave acetate, preferably 15 mg of anecortave acetate. It can also be delivered locally or topically at concentrations ranging from 0.1%-6%. The NSAls alone or in combination with anecortave acetate are useful for treating persons suffering from retinal edema, PPDR and/or NPDP The NSAIs and anecortave acetate may be formulated together and administered or formulated and administered separately. The following example is preferably administered juxtasclerally.

### EXAMPLE 3

| Ingredient | Concentration w/v % |
|---|---|
| Anecortave Acetate | 3% |
| Monobasic Sodium Phosphate Diltydrate | 0.051% |
| Dibasic Sodium Phosphate Dodecahydrate | 0.5% |
| Tyloxapol | 0.05-0.4% |
| Sodium Chloride | 0.76% |
| NaOH/HCl | pH adjust to 5.0-8.4 |
| Water for injection | q.s. 100% |

### EXAMPLE 4

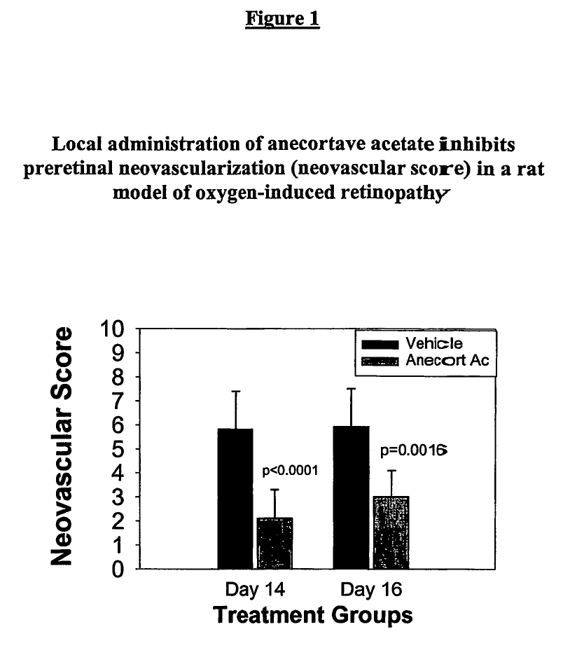

Anecortave acetate was tested for its angiostatic efficacy in a rat pup model of retinopathy of prematurity (Penn, at al., Investigative Ophthalmology & Visual Science, “The Effect of an Angiostatic Steroid on Neovascularization in a Rat Model of Retinopathy of Prematurity,” Vol. 42(1):283-290, January 2001). Newborn rat pups were placed in an atmosphere of varying oxygen content. The rats received a single intravitreal injection of vehicle or anecortave acetate (500 μg) upon return to room air (day 14) or 2 days later (day 16). There was significant retinal neovascularization in the rats that received vehicle injections. Anecortave acetate significantly inhibited retinal neovascularization by 66% and 50% on days 14 and 16 (respectively). See FIG. 1.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

I claim:

1. A method for treating pathologic ocular angiogenesis and any associated edema which comprises, administering a composition comprising an effective amount of a non-steroidal anti-inflammatory and an angiostatic agent.

2. The method of claim 1 wherein the angiostatic agent is anecortave acetate.

3. The method of claim 1, wherein the non-steroidal anti-inflammatory is nepafenac.

4. A method for treating a person suffering from retinal edema or non-proliferative diabetic retinopathy which comprises, administering an effective amount of a non-steroidal anti-inflammatory agent.

5. The method of claim 4, further comprising administering an effective amount of an angiostatic agent.

6. The method of claim 5, wherein the angiostatic agent is anecortave acetate.

7. The method of claim 4, wherein the non-steroidal anti-inflammatory agent is nepafenac.
